(19) United States
(12) Patent Application Publication
(10) Pub. No.: US 2023/0086746 A1
(43) Pub. Date: Mar. 23, 2023

(54) DIAMINE MONOMER COMPOUND, METHOD FOR PREPARING THE SAME, RESIN, FLEXIBLE FILM, AND ELECTRONIC DEVICE

(71) Applicant: Zhen Ding Technology Co., Ltd., Tayuan (TW)

(72) Inventors: KUAN-WEI LEE, Tayuan (TW); SZU-HSIANG SU, Taoyuan (TW); SHOU-JUI HSIANG, Tayuan (TW); CHING-HSUAN LIN, Taoyuan (TW); WAN-LING HSIAO, Tayuan (TW); REN-YU YEH, Tayuan (TW)

(21) Appl. No.: 17/490,108

(22) Filed: Sep. 30, 2021

(30) Foreign Application Priority Data
Aug. 25, 2021 (TW) 110131547

(51) Int. Cl. C08G 73/16; C07C 229/60; C07C 41/09; C07C 201/12; C07C 227/04; C08J 5/18; H05K 1/03

(52) U.S. Cl. CPC C08G 73/16; C07C 229/60; C07C 41/09; C07C 201/12; C07C 227/04; C08J 5/18; H05K 1/0393; H05K 1/0346; C08J 2379/08; H05K 2201/0154

(57) ABSTRACT

A diamine monomer compound with a structural formula of

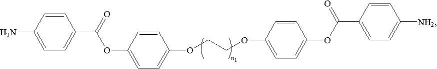

wherein n₁ is an integer greater than 1, forms the basis of a dielectric material with reduced dielectric losses for improved signals transmission. A method for preparing the compound, a polyimide resin made from the compound, a flexible film, and an electronic device including the polyimide resin are also disclosed. The compound has a long but flexible even numbered carbon chain and a liquid crystal unit structure. The reduced regularity and rigidity of the molecular chain make the polyimide resin convenient for film-forming. Dimensional stability is improved, the coefficient of thermal expansion of the materials is reduced, and the materials have good mechanical and thermal properties, the electron loss factor and coefficient of thermal expansion of the materials being reduced.

### FIELD

The subject matter herein generally relates to a diamine monomer compound, a method for manufacturing the diamine monomer, a polyimide polymer resin made from the diamine monomer compound, and a flexible film and an electronic device including the polyimide polymer resin.

### BACKGROUND

In electronic signal transmissions, loss of the transmission signal is mainly a result of dielectric loss of a dielectric layer. Dielectric loss is positively correlated with dielectric loss factor and dielectric constant. The polarity of material of the dielectric layer will affect the stability of electron transmission in a conductor. If the polarity of a molecular structure of the material of the dielectric layer is large, the electrons in the conductor will be attracted by the dielectric layer after a circuit board is polarized, which will seriously affect the stability of electron transmission. Designing the polymer structure of the dielectric layer to reduce the dielectric loss of the dielectric layer and to achieve good insulation effect is problematic.

Currently, since liquid crystalline polymer (LCP) materials have liquid crystal structure, the LCP materials have low dielectric loss and are widely used in printed circuit boards. Although the LCP materials have the liquid crystal structure which has a good forward arrangement, the film-forming property of the LCP materials is poor, the film-forming process is limited, and it is difficult to laminate a film formed from the LCP materials onto a copper plate to form a copper clad laminate.

### BRIEF DESCRIPTION OF THE DRAWINGS

Implementations of the present technology will now be described, by way of embodiment, with reference to the attached figures.

FIG. 1 is a polarizing microscope photograph of a diamine monomer according to one embodiment of the present disclosure.

FIG. 2 is a is a hydrogen spectrum of an intermediate I′ according to other embodiment of the present disclosure.

FIG. 3 is an infrared spectrum of an intermediate I′ according to another embodiment of the present disclosure.

FIG. 4 is a hydrogen spectrum of an intermediate II′ according to another embodiment of the present disclosure.

FIG. 5 is an infrared spectrum of an intermediate II′ according to another embodiment of the present disclosure.

FIG. 6 is a hydrogen spectrum of a diamine monomer according to another embodiment of the present disclosure.

FIG. 7 is an infrared spectrum of a diamine monomer according to another embodiment of the present disclosure.

FIG. 8 is a DSC spectrum of a diamine monomer according to another embodiment of the present disclosure.

FIG. 9 is a polarizing microscope photograph of a diamine monomer according to another embodiment of the present disclosure.

FIG. 10 is a hydrogen spectrum of an intermediate I′ according to yet another embodiment of the present disclosure.

FIG. 11 is a hydrogen spectrum of an intermediate II′ according to yet another embodiment of the present disclosure.

FIG. 12 is a hydrogen spectrum of a diamine monomer according to yet another embodiment of the present disclosure.

FIG. 13 is a DSC spectrum of a diamine monomer according to yet another embodiment of the present disclosure.

FIG. 14 is a polarizing microscope photograph of a diamine monomer according to yet another embodiment of the present disclosure.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough instanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein.

The term “comprising,” when utilized, means “including, but not necessarily limited to”; it specifically indicates open-ended inclusion or membership in the so-described combination, group, series, and the like. When a first component is referred to as “connecting” to a second component, it is intended that the first component may be directly connected to the second component or may be indirectly connected to the second component via a third component between them.

In order to prepare polyimides with required comprehensive properties, a rigid aromatic dianhydride and a diamine structure are generally used, thus enhancing intramolecular and intermolecular interactions. However, the film-forming processability of such polyimides is poor. In order to obtain polyimides with comprehensive properties including film-forming abilities, long carbon chains or flexible groups (such as C═O, —O—, —S—SO2-, —CH2-, —C(CH₃)₂—) are usually introduced into the main chain or the side chain of the polyimides to reduce the rigidity of the main chain, so as to reduce the glass transition temperature (Tg) and the melting point (Tm) of the polyimides. The above long carbon chains or flexible groups are usually introduced through the monomers (diamine monomers and dianhydride monomers) for synthesizing the polyimide.

One embodiment of this disclosure provides a diamine monomer compound. The molecular structure of the diamine monomer compound introduces a liquid crystal unit and a long carbon chain, which can be used to prepare a polyimide resin with good dielectric properties, good mechanical properties, heat-tolerant thermal properties, and good film-forming abilities.

The general structural formula of the diamine monomer compound is:

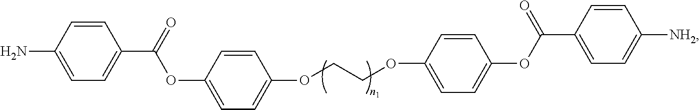

wherein n₁ is an integer greater than 1.

In some embodiments, n₁ is 2, 3, or 4.

A long carbon chain is introduced into a diamine monomer, the symmetry of the polyimide polymer and the regularity of the molecular chain are reduced due to the structural asymmetry of the long carbon chain, thus reducing the Tg and the Tm of the polyimide. The number of carbon atoms of the long carbon chain of the diamine monomer, especially the odd numbered or even numbered carbon atoms, will affect the molecular arrangement, and thus the structural form of liquid crystal. This phenomenon is called odd-even effect. Odd numbered carbon chains will make molecules more curved, have greater disorder, and need a higher temperature to form the liquid crystal phase. In addition, the formed liquid crystal is curved liquid crystal (also known as banana-shaped liquid crystal). Most of the curved liquid crystals have ferroelectricity, and the molecules of the ferroelectric materials are prone to be reversed due to electric field polarization, thus materials containing odd numbered carbon chains are mostly used in storage elements such as capacitors. Even numbered carbon chains facilitate the formation of the liquid crystal phase, such as layered liquid crystal or nematic liquid crystal. Therefore, in this disclosure, the long carbon chain structure introduced into the diamine monomer compound contains an even number of carbon atoms, and the liquid crystal units with ester groups are introduced at both ends of the even numbered carbon chain. Thus, the regularity and rigidity of the molecular chain are reduced, flexibility of the molecular chain is increased, thermal expansion coefficient is reduced, and dimensional stability is improved.

One embodiment of this disclosure provides a polyimide resin which is a condensation reaction product of the above diamine monomer compound, other aromatic or alicyclic diamine monomers different from the diamine monomer compound, and aromatic or alicyclic dianhydride monomers.

The general structural formula of the polyimide resin is:

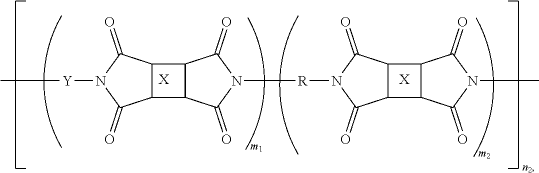

wherein X is a residue of an aromatic dianhydride or an alicyclic dianhydride, R is a residue of an aromatic diamine or an alicyclic diamine, m₁ is an integer greater than 1, m₂ is an integer greater than 1, and n₂ is an integer greater than 1. The structural formula of Y is:

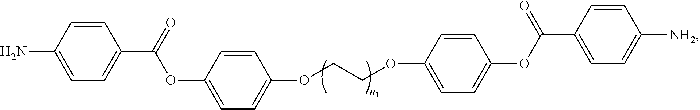

wherein n₁ is an integer greater than 1.

In the disclosure, the aromatic or alicyclic dianhydride monomer, the aromatic or alicyclic diamine monomer, and the diamine monomer compound are monomers which are polymerized to form the polyimide resin. In the structural formula of the polyimide resin, the aromatic or alicyclic dianhydride monomer and the aromatic or alicyclic diamine monomer are not present as monomer compounds, but as a group, which is defined as a residue.

The residue X of the aromatic dianhydride or the alicyclic dianhydride is selected from a group consisting of pyromellitic dianhydride, 3,3′,4,4′-biphenyl tetracarboxylic acid dianhydride, 2,3,3′,4′-biphenyl tetracarboxylic acid dianhydride, 2,3,5,6-naphthalene tetracarboxylic acid dianhydride, 2,3,6,7-naphthalene tetracarboxylic acid dianhydride, 1,4,5,8-naphthalene tetracarboxylic acid dianhydride, 2,7-dichloronaphthalene-1,4,5,8-tetracarboxylic acid dianhydride, 2,3,6,7-tetrachloron-1,4,5,8-tetracarboxylic acid dianhydride, 3,4,9,10-perylene tetracarboxylic acid dianhydride, pyrazine-2,3,5,6-tetracarboxylic acid dianhydride, thiophene-2,3,4,5-tetracarboxylic acid dianhydride, 2,3,5,6-pyridine tetracarboxylic acid dianhydride, 1,2,3,4-cyclopentane tetracarboxylic acid dianhydride, cyclobutane-1,2,3,3,4-tetracarboxylic acid dianhydride, cyclopentane-1,2,4,5-tetracarboxylic acid dianhydride, camphene-2,3,5,6-tetracarboxylic acid dianhydride, bicyclo[2.2.2]octane-7-ene-3,4,8,9-tetracarboxylic acid dianhydride, 3,3′,4,4′-benzophenone tetracarboxylic acid dianhydride, 2,2′,3,3′-benzophenone tetracarboxylic acid dianhydride, 2,3,3′,4′-benzophenone tetracarboxylic acid dianhydride, 3,3′,4,4′-diphenylsulfone tetracarboxylic acid dianhydride, 2,2′,3,3′-diphenylsulfone tetracarboxylic acid dianhydride, 2,3,3′,4′-diphenylsulfone tetracarboxylic acid dianhydride, 3,3′,4,4′-diphenylether tetracarboxylic acid dianhydride, 2,2′,3,3′-diphenylether tetracarboxylic acid dianhydride, 2,2-[bis (3,4-dicarboxyphenyl)]hexafluoropropane dianhydride, 5-(2,5-dioxo tetrahydro)-3-methyl-3-cyclohexene-1,2-dicarboxylic anhydride, and any combination thereof.

The residue R of the aromatic diamine residue or the alicyclic diamine is selected from a group consisting of 4,4′-diaminodiphenylether, 3,4′-diaminodiphenyl ether, 1,4-bis(4-aminophenoxy)benzene, o-phenylenediamine, m-phenylenediamine, p-phenylenediamine, 1,5-naphthalene diamine, 2,6-naphthalene diamine, bis(4-aminophenoxyphenyl)sulfone, bis(3-aminophenoxyphenyl)sulfone, bis(4-aminophenoxy)biphenyl, bis{4-(4-aminophenoxy)phenyl}ether, 4,4′-diaminobiphenyl, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, 2,2′-bis(trifluoromethyl)benzidine, 2,6,2′,6′-tetra(trifluoromethyl)benzidine, 2,2-bis[4-(3-Aminophenoxy)phenyl]hexafluoropropane, 2,2-bis[4-(4-aminophenoxy)phenyl]hexafluoropropane, 2,2-bis(4-anilino)hexafluoropropane, 2,2-bis(3-anilino)hexafluoropropane, 2,2-bis(3-amino-4-toluenyl)hexafluoropropane, 1,6-hexanediamine, 1,4-cyclohexanediamine, 1,3-cyclohexanediamine, 1,4-bis(aminomethyl)cyclohexane, 1,3-bis(aminomethyl)cyclohexane, 4,4′-diaminodicyclohexylmethane, 4,4′-diamino-3,3′-dimethylcyclohexylmethane, and any combination thereof.

The introduction of the long even numbered carbon chain in the structure of the polyimide resin makes the molecular chains flexible, thus the regularity and the rigidity of the molecular chains is reduced compared with traditional liquid crystal materials, the polyimide resin is convenient for film-forming. Long even numbered carbon chains and liquid crystal cells (such as ester liquid crystal cells) are introduced into the main chain, the liquid crystal cells being rigid and aligned, so that the polyimide resin has a liquid crystal morphology which has a good forward arrangement, annealing can be carried out to improve the crystallinity, the dimensional stability is improved, the materials have good mechanical and thermal properties, and the loss factor (D_{f}) and the coefficient of thermal expansion (CTE) of the materials are reduced. In addition, the long even numbered carbon chain has a hydrophobic structure and increases the flexibility of the molecular chain. The combination of the long even numbered carbon chain reduces the dielectric constant (Dₖ) and the coefficient of thermal expansion (CTE) of the materials.

One embodiment of this disclosure provides a flexible film including the above polyimide resin.

One embodiment of this disclosure provides an electronic device including a circuit board. The circuit board includes the above flexible film. The above polyimide resin has good flexibility, low polarity, and good film-forming property, thus the flexible film made from the above polyimide resin has a strong bonding force with the substrate interface, the circuit board including the flexible film has good mechanical and electrical properties. Since the polyimide resin has a low coefficient of thermal expansion, peeling, cracking, or warping of the flexible film is reduced.

One embodiment of this disclosure provides a method for preparing a diamine monomer compound including:

preparing a bisphenol compound containing an even numbered carbon chain and having a general formula of

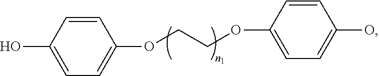

wherein n₁ is an integer greater than 1, specifically, n₁ may be 2, 3, or 4;

preparing a dinitro compound containing an even numbered carbon chain and a liquid crystal unit and having a general formula of

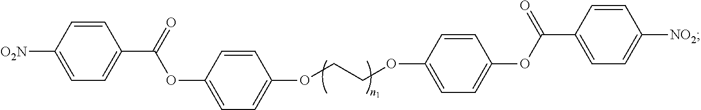

and

hydrogenating the dinitro compound to obtain the diamine monomer compound having a general formula of

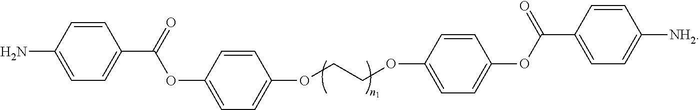

A method for preparing the above polyimide resin includes:

preparing a diamine monomer compound having a general formula of

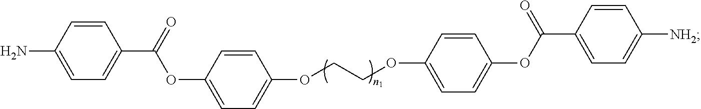

and

polymerizing the diamine monomer compound with other aromatic or alicyclic diamine monomers and aromatic or alicyclic dianhydride monomers to obtain a polyimide resin having a general formula of

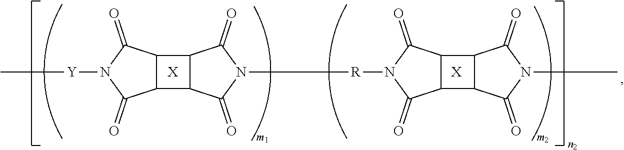

wherein X is a residue of an aromatic dianhydride residue or an alicyclic dianhydride, R is a residue of an aromatic diamine or an alicyclic diamine, m₁ is an integer greater than 1, m₂ is an integer greater than 1, and n₂ is an integer greater than 1.

In some embodiments, in the process of preparing the polyimide resin, the molar ratio of the total diamine monomers to the total dianhydride monomers is 0.9 to 1.1, and the molar ratio is preferably 1. That is, the ratio of the total moles of the diamine monomer compound and other aromatic or alicyclic diamine monomers to the total moles of the aromatic or alicyclic dianhydride monomers is 0.9 to 1.1.

In some embodiments, a molar ratio of the diamine monomer compound to the total other aromatic or alicyclic diamine monomers is 1:9 to 3:7, that is, the ratio of the mole of the diamine monomer compound to the total mole of the other diamine monomers is 1:9 to 3:7.

The present disclosure is illustrated by way of different examples.

### Example 1

Preparation of Monomer:

First step, hydroquinone (10.19 g, 9.26*10 mmol), K₂CO₃ (2.56 g, 9.26*2 mmol), and N,N-dimethylacetamide (DMAC, 15 g) were added into a 100 mL three-neck reactor to form a solution. 1,4-dibromobutane (2 g, 9.26 mmol) was slowly dropped into the solution and reacted at 55° C. for 24 hours in a nitrogen atmosphere. After the reaction, the solution was poured into water to precipitate, followed by washing and filtering to take out a filter cake. The filter cake was dried at 70° C. in a vacuum atmosphere to obtain an intermediate product I.

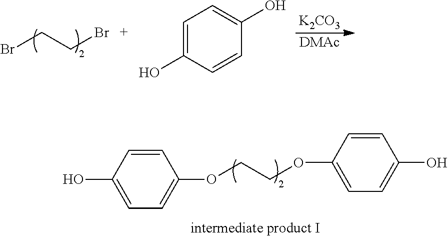

Second step, the intermediate product I (0.898 g, 3.645 mmol) and triethylamine (1.106 g, 3.645 mmol*3), and tetrahydrofuran (THF, 20 mL) were added into a 100 mL three-neck reactor. 4-nitrobenzoyl chloride (2.029 g, 3.645 mmol*3) was slowly dropped into the above three-neck reactor and reacted at 55° C. for 24 h in a nitrogen atmosphere. During the reaction, the salts of triethylamine precipitated out naturally. After the reaction, the salts of triethylamine were removed by a suction filtration method, the filtrate was collected and poured into water to precipitate, followed by washing with hot ethanol and filtering to obtain a filter cake, then the filter cake was dried at 60° C. in a vacuum environment to obtain an intermediate product II.

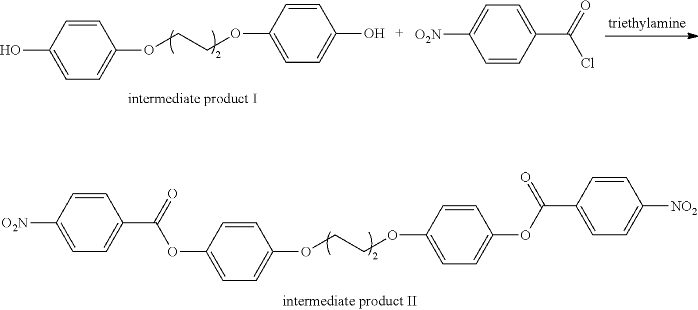

Third step, the intermediate product II (1 g, 1.837 mmol), DMF (20 mL), and palladium carbon (Pd/C, 0.04 g) were added into a 100 mL high-pressure reactor, nitrogen was injected into the reactor three times, and finally a reaction was carried out at 50° C. and 140 pa hydrogen pressure to form a solution until the hydrogen pressure stopped dropping. When the hydrogen pressure was constant, the reaction was finished. After the end of the reaction, diatomite was laid onto a ceramic funnel and the palladium carbon of the solution was removed by a suction filtration method through the ceramic funnel. The filtrate was collected and poured into deionized water to precipitate, washed with 50° C. ethanol, and filtered by the suction filtration method. The filter cake was dried at 60° C. in vacuum environment to obtain a diamine monomer compound A.

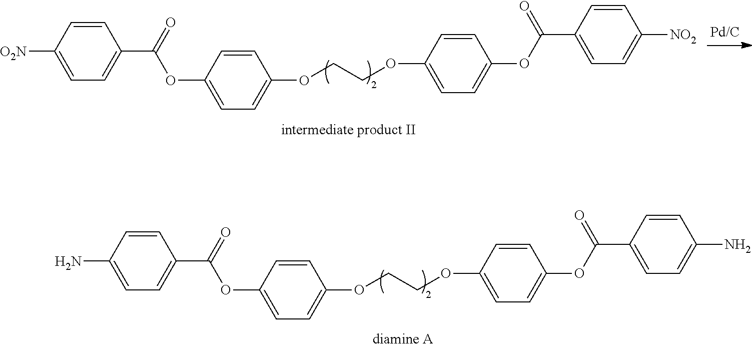

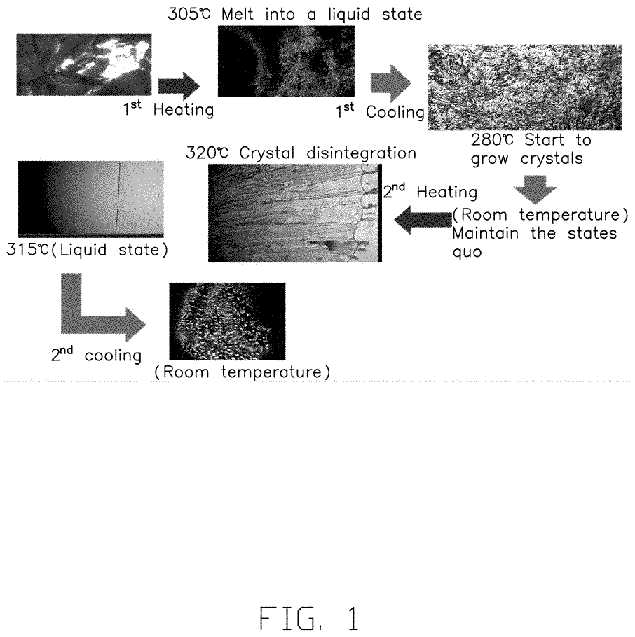

FIG. 1 is a polarizing microscope (POM) photograph of the diamine A. A heating rate of the diamine A was 10° C./min, and the diamine A at the room temperature did not have any liquid crystal phase. When the temperature reached 305° C., the diamine A melted into liquid. Then, with the decrease of temperature (cooling rate was 3° C./min), crystals began to grow at 280° C., and the liquid crystal phase was formed. With the decrease of the temperature, a large number of liquid crystal phases appeared. When the temperature reached room temperature, the liquid crystal phases was maintained. The heating was repeated. When the temperature reached 320° C., the crystallization gradually disintegrated and the liquid crystal phase gradually disappeared. When the temperature reached 315° C., the liquid crystal phases could flow slowly. It was cooled to the room temperature again, recrystallized, and the liquid crystal phase appeared. FIG. 1 shows that the diamine A with long carbon chain containing four carbon atoms can still form a liquid crystal phase during repeated heating and cooling processes. At the same time, it still maintains liquid crystal state even at the cyclization temperature. It can be seen that diamine residues with even numbered carbon chains still have a regularly arranged liquid crystal type after the polyamic acid is cyclized to form the polyimide.

### Example 2

Preparation of Monomer (with a Structure Different from the Monomer of Example 1):

First step, hydroquinone (10.19 g, 9.26*10 mmol) and K₂CO₃ (2.56 g, 9.26*2 mmol), and N,N-dimethylacetamide (DMAC, 15 g) were added into a 100 mL three-neck reactor to form a solution. 1,6-dibromohexane (2.26 g, 9.26 mmol) was slowly dropped into the solution and reacted at 55° C. for 24 hours in a nitrogen atmosphere. After the end of the reaction, the solution was poured into water to precipitate, followed by washing and filtering to take out a filter cake. The filter cake was dried at 70° C. in a vacuum atmosphere to obtain an intermediate product I′.

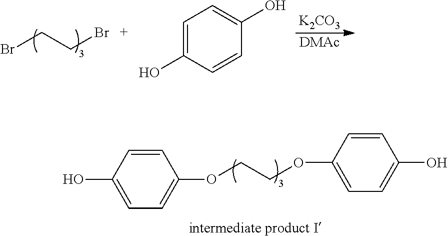

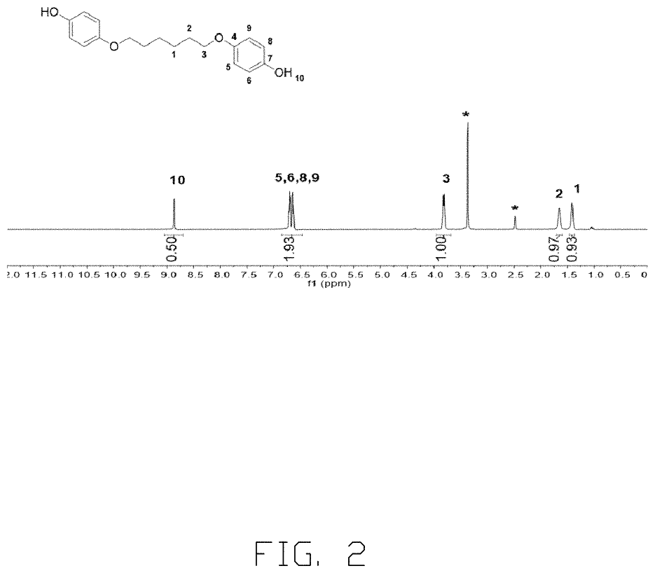

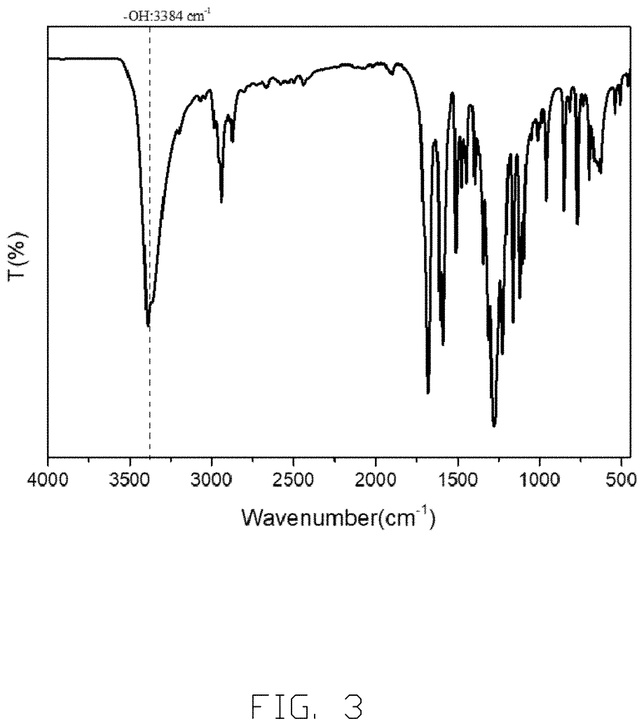

As can be seen from the hydrogen spectrum ¹H-NMR (ppm, DMSO-d₆) of FIG. 2, δ=1.40 (4H, H¹), 1.65 (4H, H²), 3.82 (4H, H³), 6.67 (4H, H⁸, H⁹), 6.73 (4H, H⁵, H⁶), 8.86 (2H, H¹⁰). It can be seen from the infrared spectrum of FIG. 3 that the signal peak of —OH appears at 3384 cm⁻¹. The results of the hydrogen spectrum and the infrared spectrum show that the bisphenol structure is successfully synthesized.

Second step, the intermediate product I′ (0.985 g, 3.645 mmol) and triethylamine (1.106 g, 3.645 mmol*3) were dissolved in tetrahydrofuran (THF, 20 mL) and put into a 100 mL three-neck reactor. 4-nitrobenzoyl chloride (2.029 g, 3.645 mmol*3) was slowly dropped into the above three-neck reactor and reacted at 55° C. for 24 h in a nitrogen atmosphere. During the reaction, the salts of triethylamine were precipitated out naturally. After the end of the reaction, the salts of triethylamine were removed by a suction filtration method, the filtrate was collected and poured into water to precipitate, followed by washing with hot ethanol and filtering to obtain a filter cake, then the filter cake was dried at 60° C. in a vacuum environment to obtain an intermediate product II′.

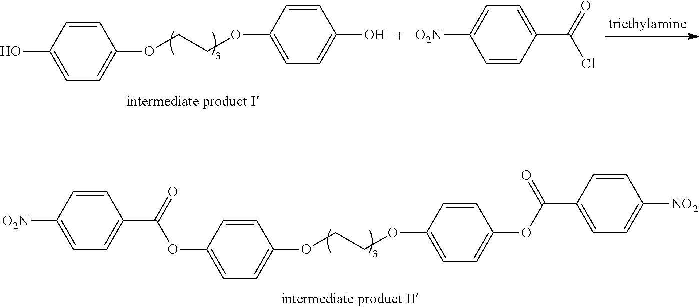

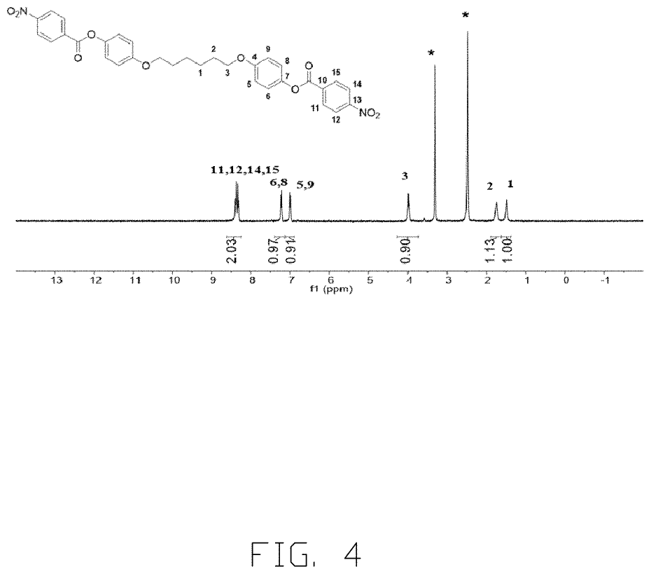

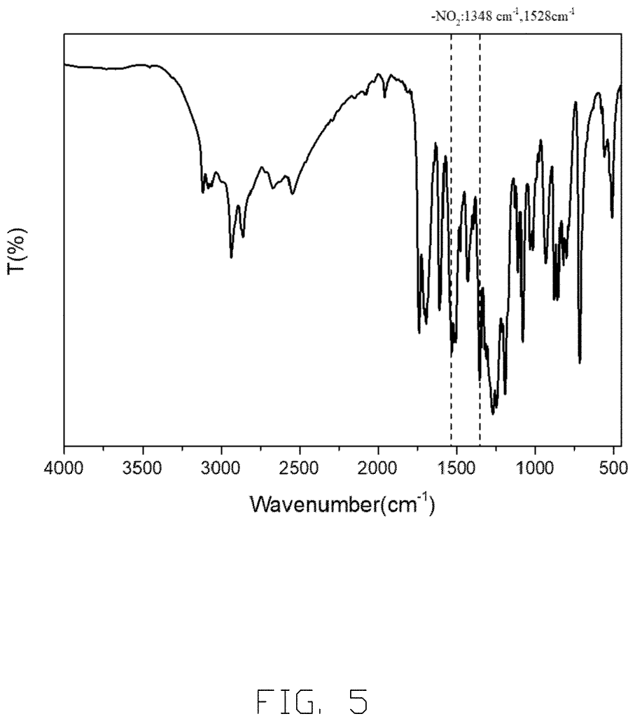

As can be seen from the hydrogen spectrum ¹H-NMR (ppm, DMSO-d6) of FIG. 4, δ=1.49 (4H, H¹), 1.74 (4H, H²), 4.00 (4H, H³), 7.00 (4H, H⁵, H⁹), 7.23 (4H, H⁶, H⁸), 8.37 (8H, H¹², H¹⁴, R¹⁵). It can be seen from the infrared spectrum (FITR) of FIG. 5 that the signal peak of —OH disappears and the signal peaks of —NO₂ appear at 1348 cm⁻¹ and 1528 cm⁻¹. The hydrogen and infrared spectra together prove that the intermediate product II′ has been successfully synthesized.

Third step, the intermediate product II′ (1 g, 1.759 mmol), DMF (20 mL), and palladium carbon (Pd/C, 0.04 g) were added into a 100 mL high-pressure reactor, nitrogen was injected into the reactor three times, and finally a reaction was carried out at 50° C. and 140 pa hydrogen pressure to form a solution until the hydrogen pressure stopped dropping. When the hydrogen pressure was constant, the reaction is over. After the end of the reaction, diatomite was laid onto a ceramic funnel and the palladium carbon of the solution was removed by a suction filtration method through the ceramic funnel. The filtrate was collected and poured into deionized water to precipitate, washed with 50° C. ethanol, and filtered by the suction filtration method. The filter cake was dried at 60° C. in vacuum environment to obtain a diamine monomer compound B.

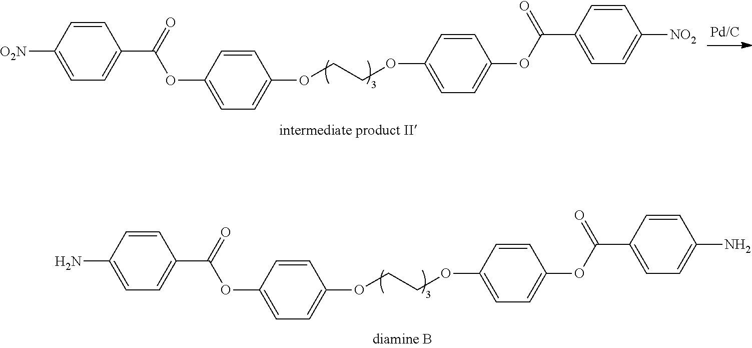

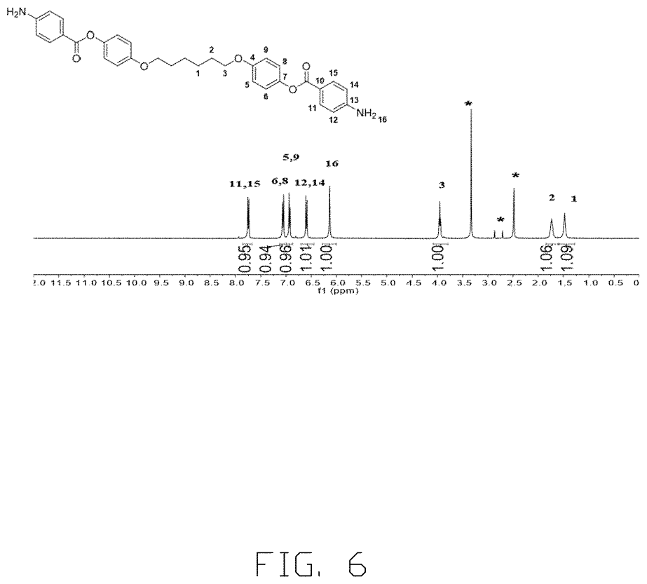

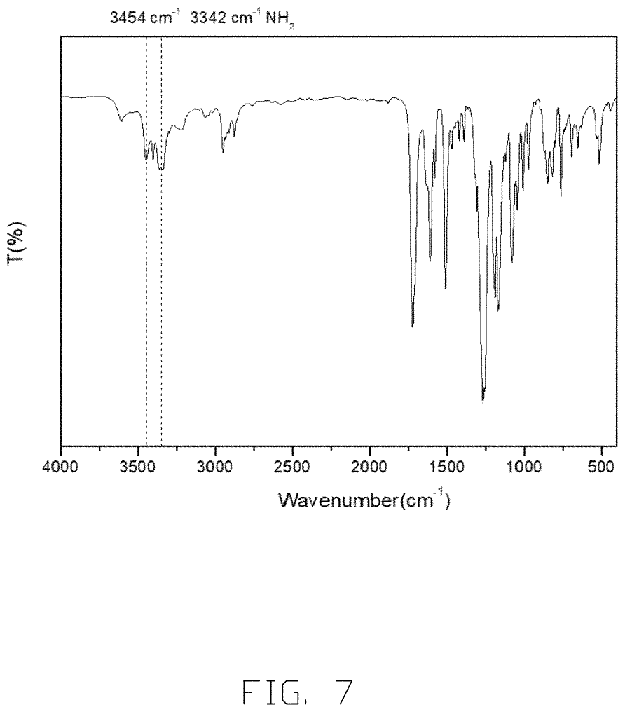

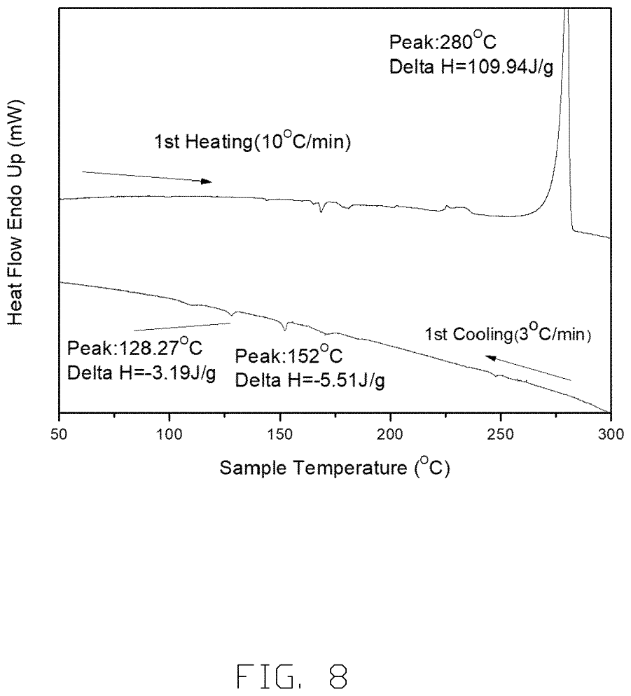

As can be seen from the hydrogen spectrum ¹H-NMR (ppm, DMSO-d6) of FIG. 6, δ=1.50 (4H, H¹), 1.85 (4H, H²), 4.05 (4H, H³), 6.16 (4H, H¹⁶), 6.61 (4H, H¹², H¹⁴), 6.92 (4H, H⁵, H⁹), 7.06 (4H, H⁶, H⁸), 7.81 (4H, H¹¹, H¹⁵). As can be seen from the infrared spectrum (FITR) of FIG. 7, the signal peaks of —NO₂ at 1348 cm⁻¹ and 1528 cm⁻¹ disappeared, resulting in the stretched vibration peaks of —NH₂ at 3356 cm⁻¹ and 3469 cm⁻¹. The hydrogen and infrared spectra together prove that the diamine B has been successfully synthesized. According to the endothermic peak in the differential scanning calorimetry (DSC) diagram of FIG. 8, the melting point ranges from 260° C. to 283° C., and the enthalpy value (Delta H) is 109.94 J/g.

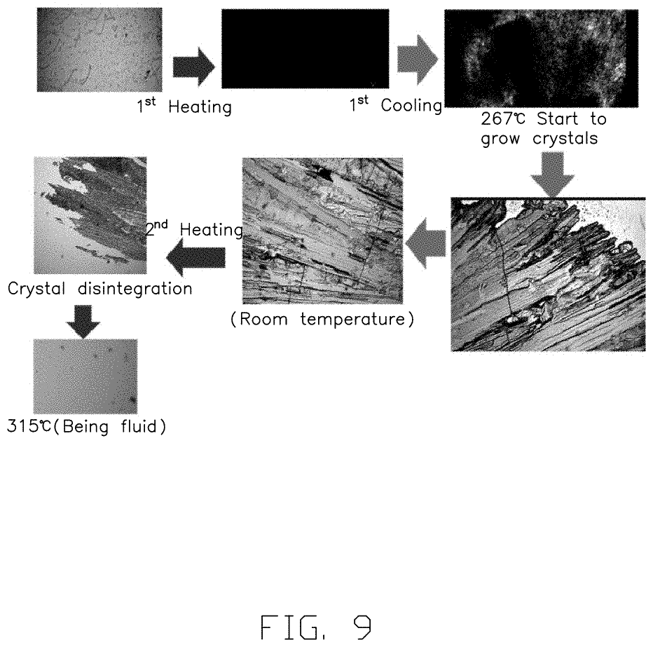

FIG. 9 is a polarizing microscope (POM) photograph of the diamine B. A heating rate of the diamine B was 10° C./min, and the diamine B at the room temperature did not have any liquid crystal phase. Heating was continued to melt the diamine B into liquid. Then, with the decrease of the temperature (cooling rate is 3° C./min), crystals began to grow at 267° C., and the liquid crystal phase was formed. With the decrease of the temperature, a large number of liquid crystal phases appeared. When the temperature reached the room temperature, the liquid crystal phases is maintained. Heat up again. When the temperature reached 320° C., the crystallization gradually disintegrated and the liquid crystal phase gradually disappeared. When the temperature reaches 315° C., the liquid crystal phases can flow slowly. It was cooled to the room temperature again, recrystallized, and the liquid crystal phase appeared. Through the comparison of DSC and POM, it can be observed that the diamine B with long carbon chain containing four carbon atoms can still form a liquid crystal phase during repeated heating and cooling processes. At the same time, it still maintained liquid crystal state even at the cyclization temperature. It can be seen that diamine residues with even numbered carbon chains still have a regularly arranged liquid crystal type after the polyamic acid is cyclized to form the polyimide.

### Example 3

Preparation of Monomer (with a Structure Different from the Monomers of Examples 1 and 2):

First step, hydroquinone (10.19 g, 9.26*10 mmol) and K₂CO₃ (2.56 g, 9.26*2 mmol), and N,N-dimethylacetamide (DMAC, 15 g) were added into a 100 mL three-neck reactor to form a solution. 1,8-dibromooctane (2.26 g, 9.26 mmol) was slowly dropped into the solution and reacted at 55° C. for 24 hours in a nitrogen atmosphere. After the end of the reaction, the solution was poured into water to precipitate, followed by washing and filtering to take out a filter cake. The filter cake was dried at 70° C. in a vacuum atmosphere to obtain an intermediate product I″.

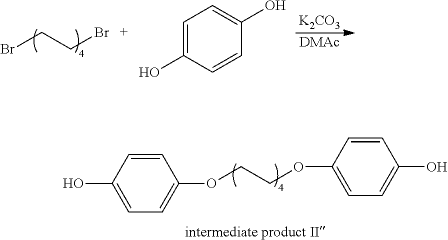

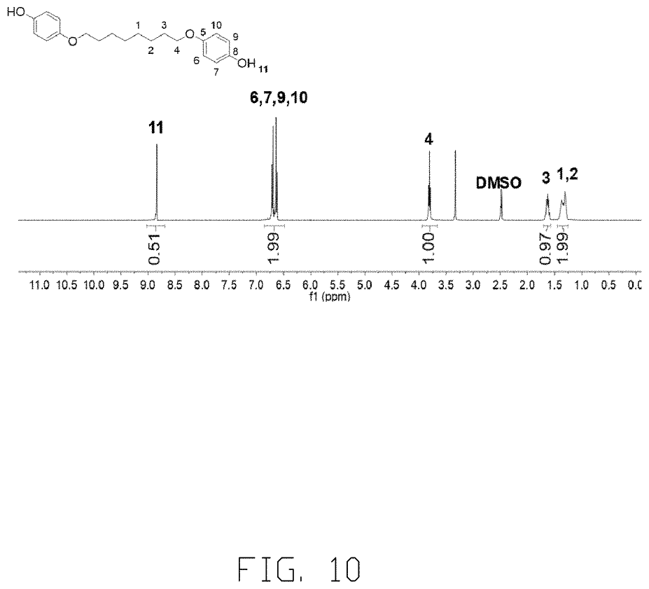

As can be seen from the hydrogen spectrum ¹H-NMR (ppm, DMSO-d₆) of FIG. 10, δ=1.33 (4 h, H¹, H²), 1.66 (2 h, H³), 3.80 (2 h, H⁴), 6.65 (4 h, H⁶, H⁷, H⁹, H¹⁰) and 8.84 (2 h, H¹¹), the bisphenol structure was successfully synthesized.

Second step, the intermediate product I″ (1.07 g, 3.645 mmol) and triethylamine (1.106 g, 3.645 mmol*3), and tetrahydrofuran (THF, 20 mL) were added into a 100 mL three-neck reactor to form a solution. 4-nitrobenzoyl chloride (2.029 g, 3.645 mmol*3) was slowly dropped into the solution and reacted at 55° C. for 24 h in a nitrogen atmosphere. During the reaction, the salts of triethylamine were precipitated out naturally. After the end of the reaction, the salts of triethylamine were removed by a suction filtration method, the filtrate was collected and poured into water to precipitate, followed by washing and filtering to obtain a filter cake, then the filter cake was dried at 60° C. in a vacuum environment to obtain an intermediate product II″.

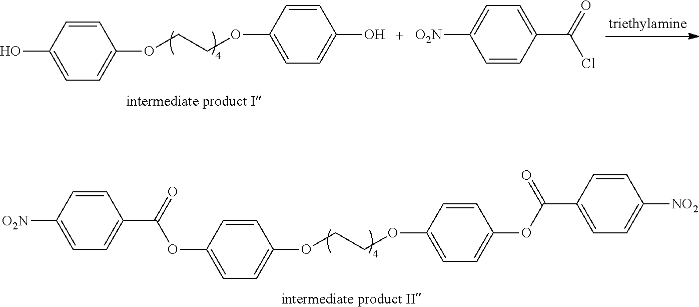

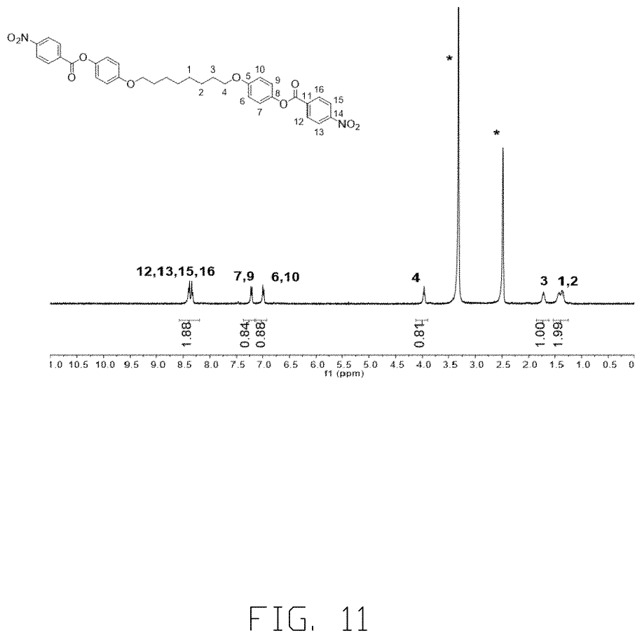

As can be seen from the hydrogen spectrum ¹H-NMR (ppm, DMSO-d6) of FIG. 11, δ=1.40 (4H, H¹, H²), 1.72 (2H, H³), 3.97 (2H, H⁴), 6.99 (4H, H⁶, H¹⁰), 7.22 (4H, H⁷, H⁹), 8.36 (8H, H¹², H¹³, H¹⁵, H¹⁶). FIG. 11 illustrates that the intermediate product II″ was successfully synthesized.

Third step, the intermediate product II″ (1 g, 1.688 mmol), DMF (20 mL), and palladium carbon (Pd/C, 0.04 g) were added into a 100 mL high-pressure reactor, nitrogen was injected into the reactor three times, and finally a reaction was carried out at 50° C. and 140 pa hydrogen pressure to form a solution until the hydrogen pressure stopped dropping. When the hydrogen pressure was constant, the reaction was over. After the end of the reaction, diatomite was laid onto a ceramic funnel and the palladium carbon of the solution was removed by a suction filtration method through the ceramic funnel. The filtrate was collected and poured into deionized water to precipitate, washed with 50° C. ethanol, and filtered by the suction filtration method. The filter cake was dried at 60° C. in vacuum environment to obtain a diamine monomer compound C.

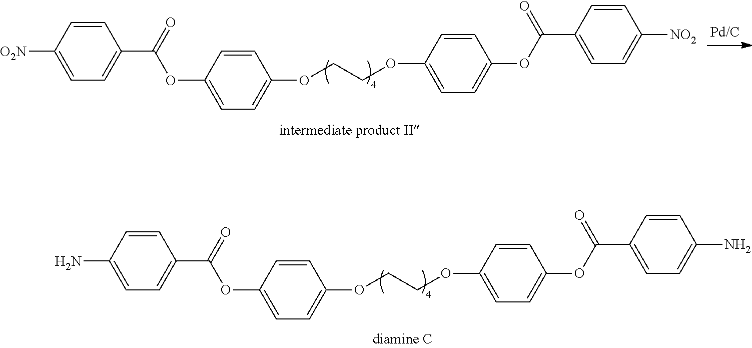

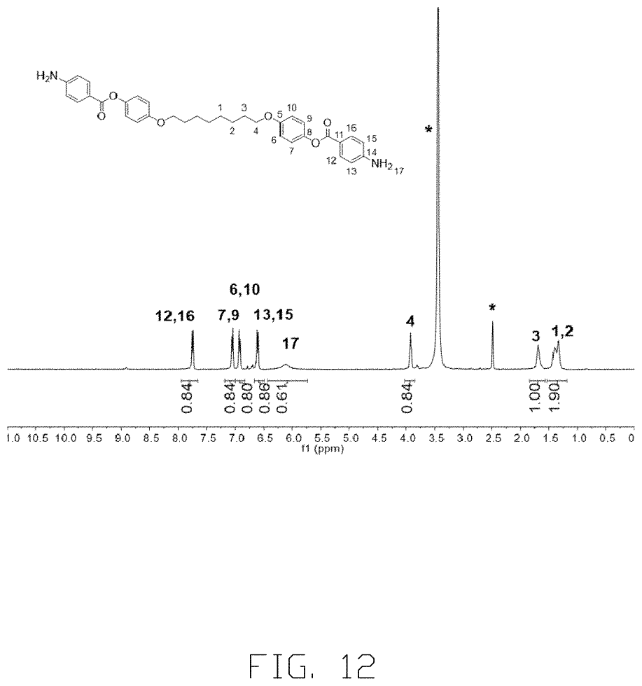

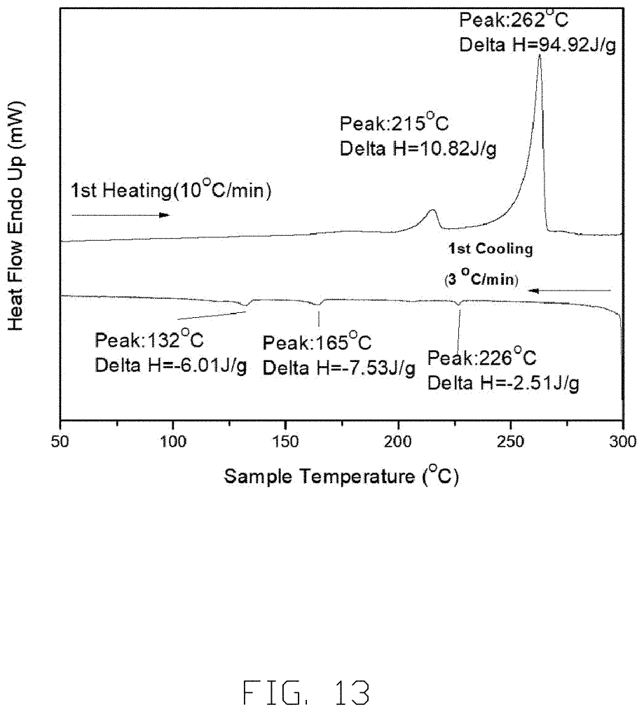

As can be seen from the hydrogen spectrum ¹H-NMR (ppm, DMSO-d6) of FIG. 12, δ=1.36 (4H, H¹, H²), 1.70 (4H, H³), 3.93 (4H, H⁴), 6.13 (2H, H¹⁷), 6.60 (4H, H¹³, H¹⁵), 6.92 (4H, H⁶, H¹⁰), 7.05 (4H, H⁷, H⁹), 7.75 (4H, H¹², H¹⁶). According to the endothermic peak in the differential scanning calorimetry (DSC) diagram of FIG. 13, the melting point ranges from 239° C. to 267° C., the melting endothermic peak is 262° C., the enthalpy value (Delta H) is 94.92 J/g, the heating rate is 10° C./min, and the cooling rate is 3° C./min.

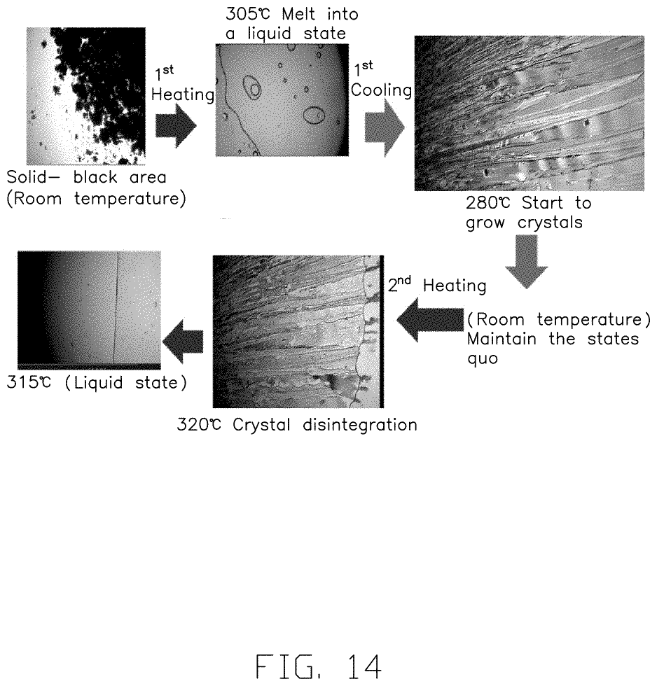

FIG. 14 is a polarizing microscope (POM) photograph of the diamine C. A heating rate of the diamine C was 10° C./min, and the diamine C at the room temperature did not have any liquid crystal phase. Continued heating to melt the diamine C into liquid was applied. Then, with the decrease of the temperature (cooling rate is 3° C./min), crystals began to grow at 280° C., and the liquid crystal phase was formed. With the decrease of the temperature, a large number of liquid crystal phases appeared. When the temperature reached the room temperature, the liquid crystal phases was maintained. Heat up again. When the temperature reached 320° C., the crystallization gradually disintegrated and the liquid crystal phase gradually disappeared. When the temperature reached 315° C., the liquid crystal phases could flow slowly. It was cooled to the room temperature again, recrystallized and the liquid crystal phase appeared. Through the comparison of DSC and POM, it can be observed that the diamine B with long carbon chain containing four carbon atoms can still form a liquid crystal phase during repeated heating and cooling processes. At the same time, liquid crystal state was maintained even at the cyclization temperature. It can be seen that diamine residues with even numbered carbon chains still have a regularly arranged liquid crystal type after the polyamic acid is cyclized to form the polyimide.

Preparation of Polymer

In the nitrogen atmosphere, the diamine A, the diamine B or the diamine C, a commercial diamine monomer (e.g. 4,4′-diaminodiphenylether, ODA), and the solvent N, N-Dimethylacetamide (DMAC) were added into a reaction bottle, followed by stirring to dissolve at the room temperature, to form a solution. Then the anhydride monomer (pyromellitic dianhydride PMDA) was slowly added into the solution and stirred at the room temperature for 24 hours to obtain a polyamic acid composition.

The polyamic acid composition was coated on a copper foil substrate and kept at a constant temperature of 100 to 150° C. for 10 to 15 minutes to remove the solvent to form a polyamic acid film. Then the polyamic acid was cyclized at 300° C. for 30-60 minutes in the nitrogen environment, to form a polyimide film with a thickness of about 12 to 50 μm, and an annealing treatment was carried out to improve the crystallinity.

According to the above DSC and POM analysis, the diamine monomer A, B, or C forms nematic liquid crystal phase at about 280° C., the crystallization begins to disintegrate at 320° C., and the liquid crystal phase remains at about 300° C. Furthermore, the diamine A, B, or C reacts with ODA and PMDA to form polyamic acid. The polyamic acid was coated on the copper foil substrate and kept at a constant temperature of 100 to 150° C. for 10 to 15 minutes to remove the solvent and to form the polyamic acid film. The acid film was subjected to a high temperature cyclization at 300° C. in an nitrogen environment to prepare the polyimide (PI) film. The prepared PI film still had the liquid crystal phase. The molecular structure of the diamine A, B, or C contained a liquid crystal unit and a long carbon chain structure with even numbered carbon atoms. Compared with the pure liquid crystal structure, the molecular flexibility of the molecular chain is increased and the polarity is decreased, conducive to reducing the loss factor (DF) and dielectric constant (DK) of the material. The existence of the long even numbered carbon chain reduced the coefficient of thermal expansion (CTE) of the material. The PI polymer has excellent film-forming performance, conducive to reducing the difficulty of film-forming of the polymer materials with liquid crystal cell structure.

The diamine monomer compound of the disclosure introduces a long even numbered carbon chain and a liquid crystal unit, the long even numbered carbon chain makes the molecular chain flexible, which reduces the regularity and rigidity of the molecular chain and facilitates processing of the polyimide resin into film-forming. Long even numbered carbon chains and liquid crystal cells (such as ester liquid crystal cells) are introduced into the main chain, the liquid crystal cells being rigid and aligned, so that the polyimide resin has a liquid crystal morphology which has a good forward arrangement, an annealing can be carried out to improve the crystallinity, the dimensional stability is improved, the materials have good mechanical and thermal properties, and the loss factor and the coefficient of thermal expansion of the materials are reduced. In addition, the long even numbered carbon chain has a hydrophobic structure and increases the flexibility of the molecular chain. The combination of the long even numbered carbon chain reduces the dielectric constant and the coefficient of thermal expansion of the materials.

While the present disclosure has been described with reference to particular embodiments, the description is illustrative of the disclosure and is not to be construed as limiting the disclosure. Therefore, those of ordinary skill in the art can make various modifications to the embodiments without departing from the scope of the disclosure as defined by the appended claims.

What is claimed is:

1. A diamine monomer compound having a structural formula of 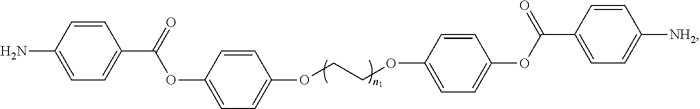
wherein n₁ is an integer greater than 1.

2. The diamine monomer of claim 1, wherein n₁ is 2, 3, or 4.

3. A polyimide resin having a structural formula of 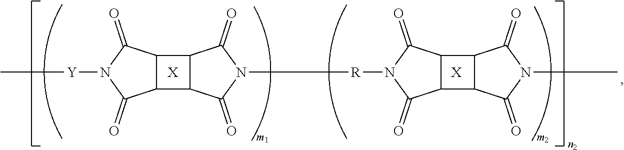
wherein X is a residue of an aromatic dianhydride residue or an alicyclic dianhydride, R is a residue of an aromatic diamine or an alicyclic diamine, m₁ is an integer greater than 1, m₂ is an integer greater than 1, and
n₂ is an integer greater than 1,
the structural formula of Y is: 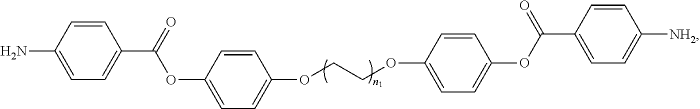
wherein n₁ is an integer greater than 1.

4. The polyimide resin of claim 3, wherein the residue X of the aromatic dianhydride or the alicyclic dianhydride is selected from a group consisting of pyromellitic dianhydride, 3,3′,4,4′-biphenyl tetracarboxylic acid dianhydride, 2,3,3′,4′-biphenyl tetracarboxylic acid dianhydride, 2,3,5,6-naphthalene tetracarboxylic acid dianhydride, 2,3,6,7-naphthalene tetracarboxylic acid dianhydride, 1,4,5,8-naphthalene tetracarboxylic acid dianhydride, 2,7-dichloronaphthalene-1,4,5,8-tetracarboxylic acid dianhydride, 2,3,6,7-tetrachloron-1,4,5,8-tetracarboxylic acid dianhydride, 3,4,9,10-perylene tetracarboxylic acid dianhydride, pyrazine-2,3,5,6-tetracarboxylic acid dianhydride, thiophene-2,3,4,5-tetracarboxylic acid dianhydride, 2,3,5,6-pyridine tetracarboxylic acid dianhydride, 1,2,3,4-cyclopentane tetracarboxylic acid dianhydride, cyclobutane-1,2,3,3,4-tetracarboxylic acid dianhydride, cyclopentane-1,2,4,5-tetracarboxylic acid dianhydride, camphene-2,3,5,6-tetracarboxylic acid dianhydride, bicyclo[2.2.2]octane-7-ene-3,4,8,9-tetracarboxylic acid dianhydride, 3,3′,4,4′-benzophenone tetracarboxylic acid dianhydride, 2,2′,3,3′-benzophenone tetracarboxylic acid dianhydride, 2,3,3′,4′-benzophenone tetracarboxylic acid dianhydride, 3,3′,4,4′-diphenylsulfone tetracarboxylic acid dianhydride, 2,2′,3,3′-diphenylsulfone tetracarboxylic acid dianhydride, 2,3,3′,4′-diphenylsulfone tetracarboxylic acid dianhydride, 3,3′,4,4′-diphenylether tetracarboxylic acid dianhydride, 2,2′,3,3′-diphenylether tetracarboxylic acid dianhydride, 2,2-[bis (3,4-dicarboxyphenyl)]hexafluoropropane dianhydride, 5-(2,5-dioxo tetrahydro)-3-methyl-3-cyclohexene-1,2-dicarboxylic anhydride, and any combination thereof.

5. The polyimide resin of claim 3, wherein the residue R of the aromatic diamine residue or the alicyclic diamine is selected from a group consisting of 4,4′-diaminodiphenylether, 3,4′-diaminodiphenylether, 1,4-bis(4-aminophenoxy)benzene, o-phenylenediamine, m-phenylenediamine, p-phenylenediamine, 1,5-naphthalene diamine, 2,6-naphthalene diamine, bis(4-aminophenoxyphenyl)sulfone, bis(3-aminophenoxyphenyl)sulfone, bis(4-aminophenoxy)biphenyl, bis {4-(4-aminophenoxy)phenyl}ether, 4,4′-diaminobiphenyl, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, 2,2′-bis(trifluoromethyl)benzidine, 2,6,2′,6′-tetra(trifluoromethyl)benzidine, 2,2-bis[4-(3-Aminophenoxy)phenyl]hexafluoropropane, 2,2-bis[4-(4-aminophenoxy)phenyl]hexafluoropropane, 2,2-bis(4-anilino)hexafluoropropane, 2,2-bis(3-anilino)hexafluoropropane, 2,2-bis(3-amino-4-toluenyl)hexafluoropropane, 1,6-hexanediamine, 1,4-cyclohexanediamine, 1,3-cyclohexanediamine, 1,4-bis(aminomethyl)cyclohexane, 1,3-bis(aminomethyl)cyclohexane, 4,4′-diaminodicyclohexylmethane, 4,4′-diamino-3,3′-dimethylcyclohexylmethane, and any combination thereof.

6. The polyimide resin of claim 3, wherein the polyimide resin is a condensation reaction product of a diamine monomer compound, other aromatic or alicyclic diamine monomers different from the diamine monomer compound, and aromatic or alicyclic dianhydride monomers, the diamine monomer has a structural formula of

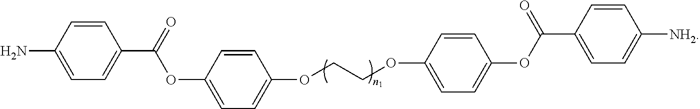

7. The polyimide resin of claim 6, wherein a ratio of a mole of the diamine monomer compound to total moles of the other aromatic or alicyclic diamine monomers is 1:9 to 3:7.

8. The polyimide film of claim 6, wherein a ratio of total moles of the diamine monomer compound and the other aromatic or alicyclic diamine monomers to total moles of the aromatic or alicyclic dianhydride monomers is 0.9 to 1.1.

9. A method for preparing a diamine monomer compound comprising:
preparing a bisphenol compound containing an even numbered carbon chain and having a general formula of 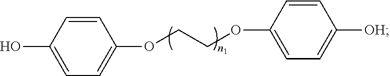
preparing a dinitro compound containing an even numbered carbon chain and a liquid crystal unit and having a general formula of 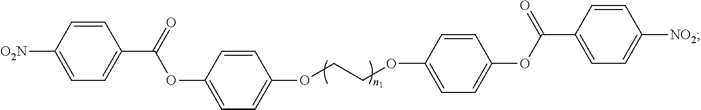
and
hydrogenating the dinitro compound to obtain the diamine monomer compound having a general formula of 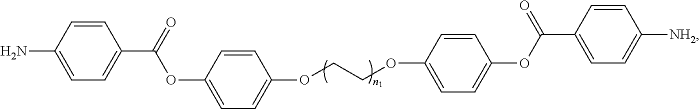
wherein n₁ is an integer greater than 1.

10. A flexible film comprising a polyimide resin, wherein the polyimide resin is having a structural formula 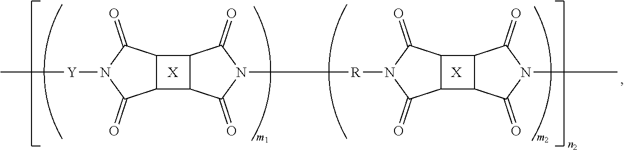
of wherein X is a residue of an aromatic dianhydride residue or an alicyclic dianhydride, R is a residue of an aromatic diamine or an alicyclic diamine, m₁ is an integer greater than 1, m₂ is an integer greater than 1, and n₂ is an integer greater than 1,
the structural formula of Y is: 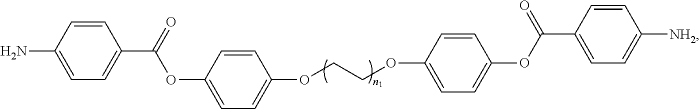
wherein n₁ is an integer greater than 1.

11. The flexible film of claim 10, wherein the residue X of the aromatic dianhydride or the alicyclic dianhydride is selected from a group consisting of pyromellitic dianhydride, 3,3′,4,4′-biphenyl tetracarboxylic acid dianhydride, 2,3,3′,4′-biphenyl tetracarboxylic acid dianhydride, 2,3,5,6-naphthalene tetracarboxylic acid dianhydride, 2,3,6,7-naphthalene tetracarboxylic acid dianhydride, 1,4,5,8-naphthalene tetracarboxylic acid dianhydride, 2,7-dichloronaphthalene-1,4,5,8-tetracarboxylic acid dianhydride, 2,3,6,7-tetrachloron-1,4,5,8-tetracarboxylic acid dianhydride, 3,4,9,10-perylene tetracarboxylic acid dianhydride, pyrazine-2,3,5,6-tetracarboxylic acid dianhydride, thiophene-2,3,4,5-tetracarboxylic acid dianhydride, 2,3,5,6-pyridine tetracarboxylic acid dianhydride, 1,2,3,4-cyclopentane tetracarboxylic acid dianhydride, cyclobutane-1,2,3,3,4-tetracarboxylic acid dianhydride, cyclopentane-1,2,4,5-tetracarboxylic acid dianhydride, camphene-2,3,5,6-tetracarboxylic acid dianhydride, bicyclo[2.2.2]octane-7-ene-3,4,8,9-tetracarboxylic acid dianhydride, 3,3′,4,4′-benzophenone tetracarboxylic acid dianhydride, 2,2′,3,3′-benzophenone tetracarboxylic acid dianhydride, 2,3,3′,4′-benzophenone tetracarboxylic acid dianhydride, 3,3′,4,4′-diphenylsulfone tetracarboxylic acid dianhydride, 2,2′,3,3′-diphenylsulfone tetracarboxylic acid dianhydride, 2,3,3′,4′-diphenylsulfone tetracarboxylic acid dianhydride, 3,3′,4,4′-diphenylether tetracarboxylic acid dianhydride, 2,2′,3,3′-diphenylether tetracarboxylic acid dianhydride, 2,2-[bis (3,4-dicarboxyphenyl)]hexafluoropropane dianhydride, 5-(2,5-dioxo tetrahydro)-3-methyl-3-cyclohexene-1,2-dicarboxylic anhydride, and any combination thereof.

12. The flexible film of claim 10, wherein the residue R of the aromatic diamine residue or the alicyclic diamine is selected from a group consisting of 4,4′-diaminodiphenylether, 3,4′-diaminodiphenylether, 1,4-bis(4-aminophenoxy)benzene, o-phenylenediamine, m-phenylenediamine, p-phenylenediamine, 1,5-naphthalene diamine, 2,6-naphthalene diamine, bis(4-aminophenoxyphenyl)sulfone, bis(3-aminophenoxyphenyl)sulfone, bis(4-aminophenoxy)biphenyl, bis{4-(4-aminophenoxy)phenyl}ether, 4,4′-diaminobiphenyl, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, 2,2′-bis(trifluoromethyl)benzidine, 2,6,2′,6′-tetra(trifluoromethyl)benzidine, 2,2-bis[4-(3-Aminophenoxy)phenyl]hexafluoropropane, 2,2-bis[4-(4-aminophenoxy)phenyl]hexafluoropropane, 2,2-bis(4-anilino)hexafluoropropane, 2,2-bis(3-anilino)hexafluoropropane, 2,2-bis(3-amino-4-toluenyl)hexafluoropropane, 1,6-hexanediamine, 1,4-cyclohexanediamine, 1,3-cyclohexanediamine, 1,4-bis(aminomethyl)cyclohexane, 1,3-bis(aminomethyl)cyclohexane, 4,4′-diaminodicyclohexylmethane, 4,4′-diamino-3,3′-dimethylcyclohexylmethane, and any combination thereof.

13. The flexible film of claim 10, wherein the polyimide resin is a condensation reaction product of a diamine monomer compound, other aromatic or alicyclic diamine monomers different from the diamine monomer compound, and aromatic or alicyclic dianhydride monomers, the diamine monomer having a structural formula of

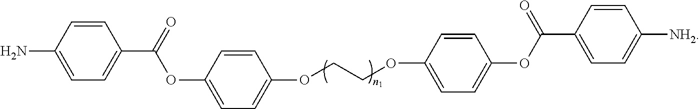

14. The flexible film of claim 13, wherein a ratio of a mole of the diamine monomer compound to total moles of the other aromatic or alicyclic diamine monomers is 1:9 to 3:7.

15. The flexible film of claim 13, wherein a ratio of total moles of the diamine monomer compound and the other aromatic or alicyclic diamine monomers to total moles of the aromatic or alicyclic dianhydride monomers is 0.9 to 1.1.

16. An electronic device comprising a circuit board, the circuit board comprising a flexible film comprising a polyimide resin having a structural formula of 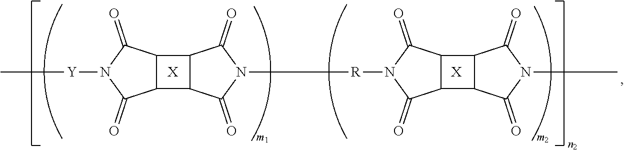
wherein X is a residue of an aromatic dianhydride residue or an alicyclic dianhydride, R is a residue of an aromatic diamine or an alicyclic diamine, m₁ is an integer greater than 1, m₂ is an integer greater than 1, and n₂ is an integer greater than 1,
the structural formula of Y is: 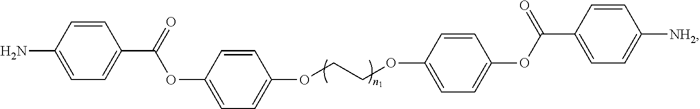
wherein n₁ is an integer greater than 1.

17. The electronic device of claim 16, wherein the residue X of the aromatic dianhydride or the alicyclic dianhydride is selected from a group consisting of pyromellitic dianhydride, 3,3′,4,4′-biphenyl tetracarboxylic acid dianhydride, 2,3,3′,4′-biphenyl tetracarboxylic acid dianhydride, 2,3,5,6-naphthalene tetracarboxylic acid dianhydride, 2,3,6,7-naphthalene tetracarboxylic acid dianhydride, 1,4,5,8-naphthalene tetracarboxylic acid dianhydride, 2,7-dichloronaphthalene-1,4,5,8-tetracarboxylic acid dianhydride, 2,3,6,7-tetrachloron-1,4,5,8-tetracarboxylic acid dianhydride, 3,4,9,10-perylene tetracarboxylic acid dianhydride, pyrazine-2,3,5,6-tetracarboxylic acid dianhydride, thiophene-2,3,4,5-tetracarboxylic acid dianhydride, 2,3,5,6-pyridine tetracarboxylic acid dianhydride, 1,2,3,4-cyclopentane tetracarboxylic acid dianhydride, cyclobutane-1,2,3,3,4-tetracarboxylic acid dianhydride, cyclopentane-1,2,4,5-tetracarboxylic acid dianhydride, camphene-2,3,5,6-tetracarboxylic acid dianhydride, bicyclo[2.2.2]octane-7-ene-3,4,8,9-tetracarboxylic acid dianhydride, 3,3′,4,4′-benzophenone tetracarboxylic acid dianhydride, 2,2′,3,3′-benzophenone tetracarboxylic acid dianhydride, 2,3,3′,4′-benzophenone tetracarboxylic acid dianhydride, 3,3′,4,4′-diphenylsulfone tetracarboxylic acid dianhydride, 2,2′,3,3′-diphenylsulfone tetracarboxylic acid dianhydride, 2,3,3′,4′-diphenylsulfone tetracarboxylic acid dianhydride, 3,3′,4,4′-diphenylether tetracarboxylic acid dianhydride, 2,2′,3,3′-diphenylether tetracarboxylic acid dianhydride, 2,2-[bis (3,4-dicarboxyphenyl)]hexafluoropropane dianhydride, 5-(2,5-dioxo tetrahydro)-3-methyl-3-cyclohexene-1,2-dicarboxylic anhydride, and any combination thereof.

18. The electronic device of claim 16, wherein the residue R of the aromatic diamine residue or the alicyclic diamine is selected from a group consisting of 4,4′-diaminodiphenylether, 3,4′-diaminodiphenylether, 1,4-bis(4-aminophenoxy)benzene, o-phenylenediamine, m-phenylenediamine, p-phenylenediamine, 1,5-naphthalene diamine, 2,6-naphthalene diamine, bis(4-aminophenoxyphenyl)sulfone, bis(3-aminophenoxyphenyl)sulfone, bis(4-aminophenoxy)biphenyl, bis{4-(4-aminophenoxy)phenyl}ether, 4,4′-diaminobiphenyl, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, 2,2′-bis(trifluoromethyl)benzidine, 2,6,2′,6′-tetra(trifluoromethyl)benzidine, 2,2-bis[4-(3-Aminophenoxy)phenyl]hexafluoropropane, 2,2-bis[4-(4-aminophenoxy)phenyl]hexafluoropropane, 2,2-bis(4-anilino)hexafluoropropane, 2,2-bis(3-anilino)hexafluoropropane, 2,2-bis(3-amino-4-toluenyl)hexafluoropropane, 1,6-hexanediamine, 1,4-cyclohexanediamine, 1,3-cyclohexanediamine, 1,4-bis(aminomethyl)cyclohexane, 1,3-bis(aminomethyl)cyclohexane, 4,4′-diaminodicyclohexylmethane, 4,4′-diamino-3,3′-dimethylcyclohexylmethane, and any combination thereof.

19. The electronic device of claim 16, wherein the polyimide resin is a condensation reaction product of a diamine monomer compound, other aromatic or alicyclic diamine monomers different from the diamine monomer compound, and aromatic or alicyclic dianhydride monomers, the diamine monomer having a structural formula of

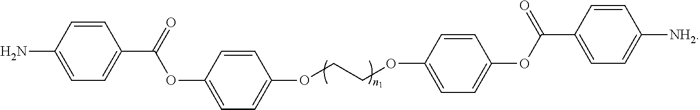

20. The electronic device of claim 19, wherein a ratio of a mole of the diamine monomer compound to total moles of the other aromatic or alicyclic diamine monomers is 1:9 to 3:7.
